(19) United States
(12) Patent Application Publication
(10) Pub. No.: US 2026/0171246 A1
(43) Pub. Date: Jun. 18, 2026

(54) DIGITAL LIFESTYLE INTERVENTION SYSTEM USING MACHINE LEARNING AND REMOTE MONITORING DEVICES

(71) Applicant: The Regents of the University of California, Oakland, CA (US)

(72) Inventors: Sujit Dey, San Diego, CA (US); Jared Johann Leitner, San Diego, CA (US); Po-Han Chiang, San Diego, CA (US)

(21) Appl. No.: 19/127,468

(22) PCT Filed: Nov. 6, 2023
(86) PCT No.: PCT/US2023/078855

§ 371 Date: May 6, 2025

Related U.S. Application Data
(60) Provisional application No. 63/382,623, filed on Nov. 7, 2022.

(51) Int. Cl. G16H 50/20; G06N 20/00; G16H 20/00; G16H 50/70

(52) U.S. Cl. CPC G16H 50/20; G16H 20/00; G16H 50/70; G06N 20/00

(57) ABSTRACT

Systems and methods for a digital lifestyle intervention system using machine learning and remote monitoring devices is described herein. The disclosed systems can include a processor configured to train, based on historical user data, a personal machine learning model to generate an output indicative of a blood pressure prediction and lifestyle feature impact on blood pressure prediction. The trained personal machine learning model can be further configured to receive user data including blood pressure data for the user and generate output including lifestyle feature impact and a blood pressure prediction by applying the trained personal machine learning model to the received user data. At least one lifestyle recommendation can be generated based on the output of the trained personal machine learning model and/or output from a population model applied to the received user data. The at least one lifestyle recommendation can be provided to a user via a user interface.

### CROSS REFERENCE TO RELATED APPLICATIONS

This application claims the benefit of and priority to U.S. Provisional Application No. 63/382,623, filed on Nov. 7, 2022, the contents of which are hereby incorporated in its entirety by reference.

### TECHNICAL FIELD

The subject matter described herein relates to a machine learning based system for lifestyle intervention.

### BACKGROUND

The United States Center for Disease Control estimates that nearly half of adults in the United States have hypertension or high blood pressure, and only about one in four adults having hypertension have their blood pressure under control. Lifestyle interventions (e.g., diet recommendations, limit on alcohol, physical activity, manage stress) are often provided to patients. Yet, lifestyle interventions are often ineffective at helping patients control blood pressure because patients are non-compliant with provided lifestyle interventions. Additionally, different lifestyle interventions may have different impacts on an individual's blood pressure, so the recommended lifestyle intervention may not be the most efficient way to reduce a person's blood pressure.

### SUMMARY

Disclosed is a machine learning based system for lifestyle intervention for improved control and monitoring of blood pressure or hypertension.

In some embodiments, a method may include the steps of training, based on historical user data, a personal machine learning model to generate an output indicative of a blood pressure prediction and data characterizing one or more lifestyle features impacting the blood pressure prediction, wherein the historical user data comprises historical user blood pressure data and historical user lifestyle data for a user; receiving, by the trained personal machine learning model, user data, wherein the user data further comprises blood pressure data for the user; generating, by the trained personal machine learning model, an output by applying the trained personal machine learning model to the received user data; generating at least one lifestyle recommendation based on at least one of the generated output from the trained personal machine learning model and output from a population model applied to the received user data; and providing at least one of the at least one generated lifestyle recommendation, and the output indicative of the blood pressure prediction, and the data characterizing one or more lifestyle features impacting the blood pressure prediction to the user via a user interface.

Optionally, the user data can include at least one of user lifestyle data, user contextual data, and user compliance data. The trained machine learning model may include at least one of a random forest model, a gradient boosting model, and a neural network. Generating the at least one recommendation for a lifestyle modification can include identifying a lifestyle recommendation from a lifestyle recommendation database communicatively coupled to the trained personal machine learning model, based on the data characterizing one or more lifestyle features impacting the blood pressure prediction. Data characterizing one or more lifestyle features impacting the blood pressure prediction can include at least one of ranking data for the one or more lifestyle features impacting the blood pressure prediction, and correlation data for the one or more lifestyle features impacting the blood pressure prediction. The population model can be configured to generate the at least one recommendation based on an aggregation of feature importance data based on a historical population. The population can include a machine learning model trained on historical population data comprising historical user data comprising historical user blood pressure data and historical user lifestyle data for a plurality of users. Further, the method can include the steps of receiving at least one of user preference data and user compliance data; and modifying the generated at least one recommendation based on at least one of user preference data and user compliance data. The method can also include the step of providing at least one of the at least one recommendation or sensor data to a clinician via a clinician user interface. The user data can include lifestyle data. Further, the method can include the steps of receiving at least one of blood pressure data, compliance data, preference data, or an updated user lifestyle data; and updating the trained machine learning model.

In some embodiments, a method can include the steps of receiving, by at least one processor, historical user data comprising historical user blood pressure data and historical user lifestyle data for a user; and training a machine learning model configured to generate an output indicative of a blood pressure prediction and data characterizing one or more lifestyle features impacting the blood pressure prediction.

Optionally, the data characterizing one or more lifestyle features impacting the blood pressure prediction can include at least one of ranking data for the one or more lifestyle features impacting the blood pressure prediction, and correlation data for the one or more lifestyle features impacting the blood pressure prediction. The machine learning model can further include at least one of a random forest model, a gradient boosting model, and a neural network. The method can further include the steps of receiving at least one of blood pressure data, compliance data, preference data, or an updated user lifestyle data; and updating the trained machine learning model.

In some embodiments, a system can include at least one data processor; and at least one memory storing instructions which, when executed by the at least one data processor, result in operations including: training, based on historical user data, a personal machine learning model to generate an output indicative of a blood pressure prediction and data characterizing one or more lifestyle features impacting the blood pressure prediction, wherein the historical user data comprises historical user blood pressure data and historical user lifestyle data for a user; receiving, by the trained personal machine learning model, user data, wherein the user data further comprises blood pressure data for the user; generating, by the trained personal machine learning model, an output by applying the trained personal machine learning model to the received user data; generating at least one lifestyle recommendation based on at least one of the generated output from the trained personal machine learning model and output from a population model applied to the received user data; and providing at least one of the at least one generated lifestyle recommendation, and the output indicative of the blood pressure prediction, and the data characterizing one or more lifestyle features impacting the blood pressure prediction, to the user via a user interface.

Optionally, the user data can include at least one of user lifestyle data, user contextual data, and user compliance data. The trained machine learning model can include at least one of a random forest model and a gradient boosting model. Generating the at least one recommendation for a lifestyle modification can include the step of identifying a lifestyle recommendation from a lifestyle recommendation database communicatively coupled to the at least one processor, based on the data characterizing one or more lifestyle features impacting the blood pressure prediction. The data characterizing one or more lifestyle features impacting the blood pressure prediction further can include at least one of ranking data for the one or more lifestyle features impacting the blood pressure prediction, and correlation data for the one or more lifestyle features impacting the blood pressure prediction. The population model can include at least one of: an aggregator configured to generate the at least one recommendation based on an aggregation of feature importance data based on a historical population; or a machine learning model trained on historical population data comprising historical user data comprising historical user blood pressure data and historical user lifestyle data for a plurality of users. The operations can also include: receiving, by the at least one processor, at least one of user preference data and user compliance data; and modifying, by the at least one processor, the generated at least one recommendation based on at least one of user preference data and user compliance data. The operations can also include receiving at least one of blood pressure data, compliance data, preference data, or an updated user lifestyle data; and updating the trained machine learning model.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a system block diagram for a machine learning based lifestyle monitoring and intervention system, in accordance with some embodiments of the present disclosure.

FIG. 2 is a process flow diagram for a machine learning based lifestyle monitoring and intervention system, in accordance with some embodiments of the present disclosure.

FIG. 3 is a system block diagram for a lifestyle feature engineering model of a machine learning based lifestyle monitoring and intervention system, in accordance with some embodiments of the present disclosure.

FIG. 4 is a system block diagram for a personal machine learning model of a machine learning based lifestyle monitoring and intervention system, in accordance with some embodiments of the present disclosure.

FIG. 5 is a system block diagram for a population model of a machine learning based lifestyle monitoring and intervention system, in accordance with some embodiments of the present disclosure.

FIG. 6A is a system block diagram for an implementation of a machine learning based lifestyle monitoring and intervention system, in accordance with some embodiments of the present disclosure.

FIG. 6B is a system block diagram for an implementation of a machine learning based lifestyle monitoring and intervention system, in accordance with some embodiments of the present disclosure.

FIG. 6C is a system block diagram for an implementation of a machine learning based lifestyle monitoring and intervention system, in accordance with some embodiments of the present disclosure.

FIG. 7 is a system block diagram for a lifestyle intervention recommendation model of a machine learning based lifestyle monitoring and intervention system, in accordance with some embodiments of the present disclosure.

FIG. 8 is a system block diagram for a hyper-personalization model of a machine learning based lifestyle monitoring and intervention system, in accordance with some embodiments of the present disclosure.

FIG. 9 is a system block diagram for a clinician notification module of a machine learning based lifestyle monitoring and intervention system, in accordance with some embodiments of the present disclosure.

FIG. 10A is an diagram of a user interface of a machine learning based lifestyle monitoring and intervention system, in accordance with some embodiments of the present disclosure.

FIG. 10B is an diagram of a user interface of a machine learning based lifestyle monitoring and intervention system, in accordance with some embodiments of the present disclosure.

FIG. 10C is an diagram of a user interface of a machine learning based lifestyle monitoring and intervention system, in accordance with some embodiments of the present disclosure.

FIG. 10D is a diagram of a user interface of a machine learning based lifestyle monitoring and intervention system, in accordance with some embodiments of the present disclosure.

FIG. 11 is a schematic diagram of precise and hyper-personalized recommendations provided by a machine learning based lifestyle monitoring and intervention system, in accordance with some embodiments of the present disclosure.

FIG. 12 is a system diagram for a machine learning based lifestyle monitoring and intervention system, in accordance with some embodiments of the present disclosure.

Like labels are used to refer to same or similar items in the drawings.

### DETAILED DESCRIPTION

Disclosed herein is a digital lifestyle monitoring and intervention system which uses machine learning (ML) to provide improved control and monitoring of blood pressure (i.e., hypertension). The disclosed systems may generate lifestyle recommendations to help a patient manage and control their blood pressure based on the lifestyle interventions most likely to have the greatest impact on controlling and managing the patient's blood pressure, the patient's lifestyle preferences, and health goals. The lifestyle recommendations can be generated using the output from a machine learning (ML) based model.

In some embodiments, the disclosed digital lifestyle monitoring and intervention system can collect multi-dimensional data and apply one or more machine learning based models to generate output which can then be used to determine lifestyle recommendations. For example, one or two lifestyle recommendations can be provided to a user or patient. In some embodiments, the provided lifestyle recommendations can be targeted interventions that are specifically tailored to the user and may provide effective control and management of blood pressure for that particular user. In this way, the disclosed machine learning (ML) based models can be used for precise and personalized recommendations. The disclosed machine learning (ML) based model(s) can evolve over time in order to capture the dynamic relationship between a user or patient's lifestyle and their hypertension.

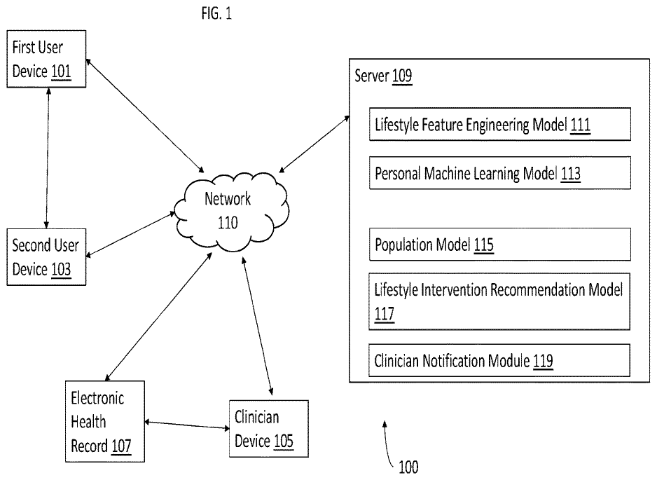

FIG. 1 illustrates an example system for a digital lifestyle monitoring and intervention. As illustrated in FIG. 1, a digital lifestyle monitoring and intervention system 100 may include a plurality of user devices 101, 103, that are communicatively coupled to server 109 which may include at least one processor via network 110. Optionally, the system components 100 may be communicatively coupled to at least one provider or clinician device 105 and/or an electronic health record system 107.

The user devices 101, 103 can be configured for data collection, receiving data, and/or display of a personalized recommendations to a user. For example, user devices 101, 103, may include a first user device 101 configured for data collection. Examples of user devices 101,103 may include blood pressure monitors, activity trackers, wearable devices, smart watches, mobile phones, tablets, and the like. Blood pressure monitors may include automatic blood pressure cuffs, manual blood pressure cuffs, or the like. The user devices 101 may be configured to record or receive user data.

User devices 101, 103 can include a second user device 103 also configured for data collection and/or data aggregation. The second user device 103 can be configured to receive user data from a plurality of first user devices 101. For example, a second user device 103 may receive blood pressure data from a blood pressure cuff and step and heart rate data from an activity tracking device. The second user device 103 may include a software application, graphical user interface, or the like, that is configured to receive user data. For example, a software application can be provided on a mobile device that is configured to present a user with a questionnaire and record user responses to the questionnaire. Examples of second user device 103 include a smart phone, tablet, laptop, desktop computer, and the like.

Although two user devices are illustrated in FIG. 1, it is envisioned that any number of suitable devices may be used in connection with the description herein. For example, a user may have a plurality of first user devices 101 configured to generate and/or record user data. Additionally, a user may have a plurality of second user devices 103 configured to receive data generated by the first user devices 101 and/or from user input to a software application on the second user device 103. For example, a user may use a manual blood pressure cuff and input blood pressure cuff readings and/or lifestyle data into a questionnaire provided via a software application on the second user device 103. User devices 101, 103 may also be communicatively coupled by Bluetooth®, wireless transmission, or any other suitable means.

User data generated, recorded, and received by user devices 101, 103 may include blood pressure data, user lifestyle data, contextual user data, and user compliance data. For example, blood pressure data may include one or more blood pressure readings over a time range. In some embodiments, the blood pressure reading may include a measure of a user's systolic and/or diastolic pressure, along with a timestamp representative of when the blood pressure reading was obtained. In some embodiments, blood pressure data may include multiple blood pressure readings. User lifestyle data may include data that indicative of user activity, routines, behaviors, choices, and practices that may be impactful on user physical health and well-being including blood pressure. For example, user lifestyle data may include daily activities, including work, exercise, and leisure, as well as aspects like sleep, diet and nutrition, and mental and emotional well-being including stress and mood. Additionally, user lifestyle data can include health practices such as taking medications. For example, user lifestyle data may include sleep, activity levels, diet, stress, mood, and the like. Sleep data may include duration of sleep, quality of sleep, timing of sleep, number of wakes, and the like. Activity level data may include number of steps taken, amount of time performing intensive cardiovascular activity, heart rate, heart rate variability, flights of steps climbed, and the like. Diet data may include caloric intake, sodium intake, nutritional profile of food ingested, and the like. Mood data may include a self-assessment of mood and emotional well-being. For example, users may rate their current mood on a rating scale used to measure opinions, attitudes, or behaviors (e.g., 1-5 Likert Scale). Stress data may include a self-assessment of stress level. For example, users may also rate their current stress on a rating scale (e.g., 1-5 Likert Scale).

The user data can also include user contextual data. This may include user demographic data and/or user medication data. For example, user demographic data may include a user's preferred gender, biological sex, age, height, weight, ethnicity, and the like. The user medication data may include a record of medications prescribed to the user for management of hypertension and/or a medication log indicating whether a user has taken their prescribed medications as indicated.

The user data can also include user compliance data. User compliance data may include data indicative of user preferences. User preferences can be indicative of whether a user would be amenable to making certain lifestyle changes, or if they would prefer making one category of lifestyle change over another. For example, a user can indicate whether they would prefer to not receive a certain lifestyle recommendation such as improving sleep hygiene or reducing sodium consumption. User compliance data may also include lifestyle changes data indicative of whether the user has taken action in conformity with lifestyle recommendations previously provided by the system. For example, if the lifestyle recommendation was to increase step count, compliance data may include the user's change in step count from before to after the recommendation was given.

In some embodiments, user preference data can be collected after a lifestyle recommendation has already been provided to a user. For example, a few days after providing a user with a lifestyle recommendation, an application on a user device may ask the user how they are feeling, their compliance with the recommendation, and whether they would like to see the previously provided lifestyle recommendation again. A user can then provide feedback to the disclosed system by way of user compliance data and/or user preference data via the user interface.

In some embodiments, the user preference data can include user hyper-personalization data. For example, when a lifestyle recommendation is provided to a user (e.g., to improve sleep hygiene) a user can provide an indication as to how they would prefer to follow that recommendation (e.g., by avoiding large meals).

User data can be generated, recorded, and/or received by user devices 101, 103 on any suitable time interval. For example, user data such as contextual data may be updated once. User data such as activity tracker data may be updated every 1 minute, 5 minutes, 30 minutes, 1 hour, 2 hours, 10 hours, 24 hours, etc. User contextual data pertaining to medications may be updated every month, two months, visit to the doctor's office, etc.

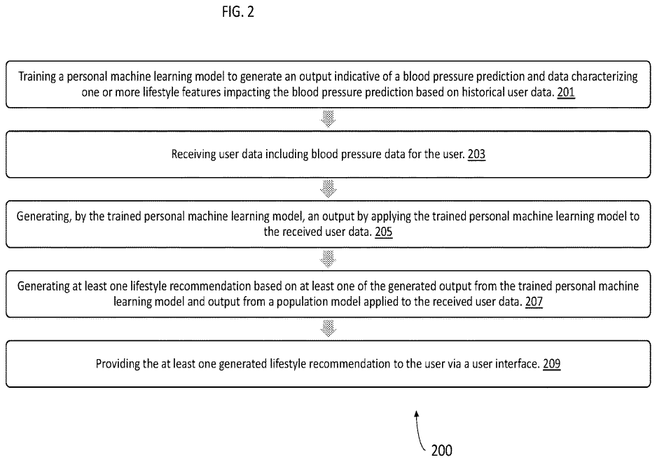

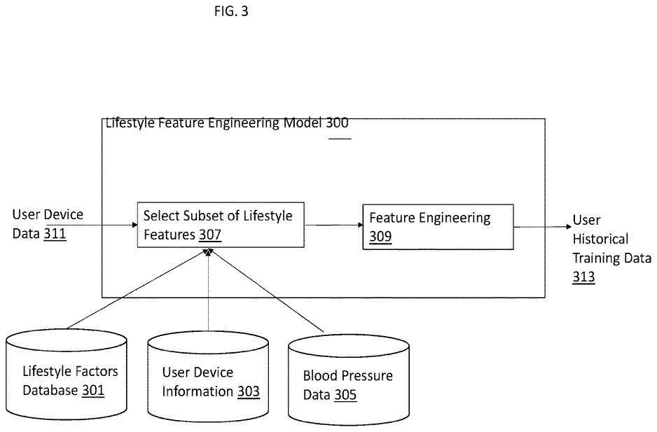

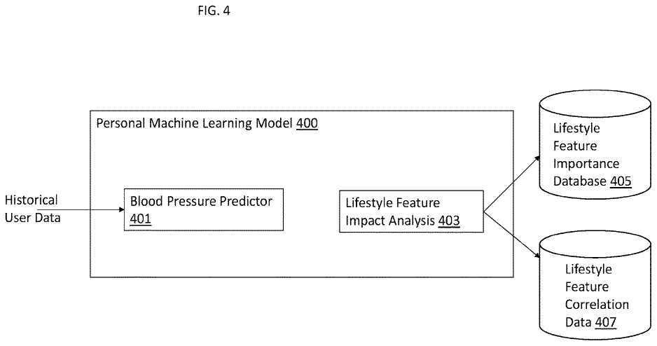

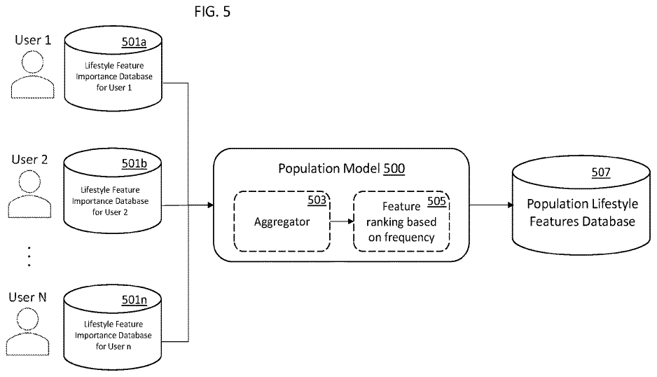

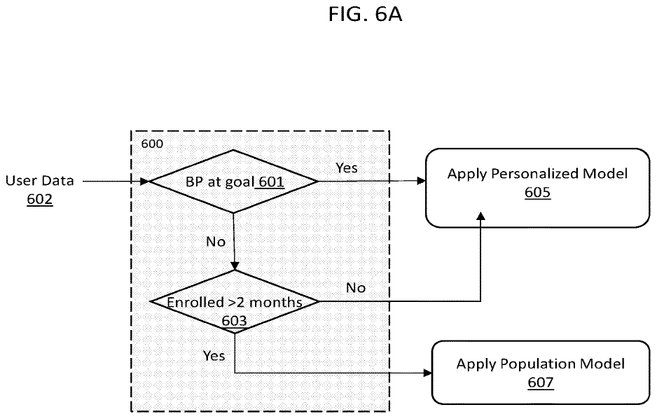

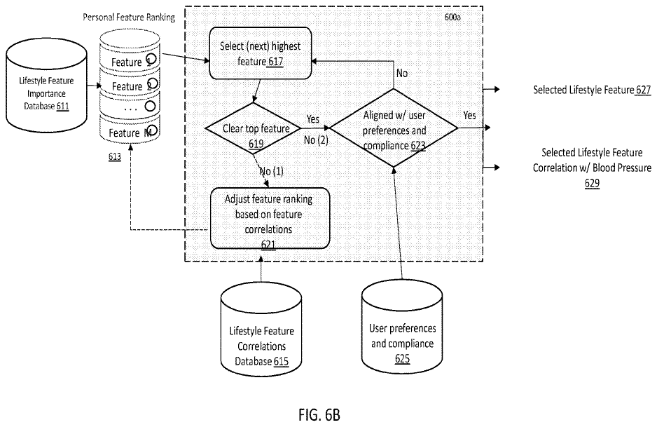

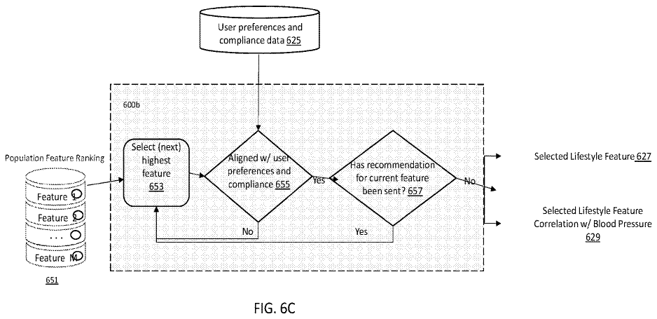

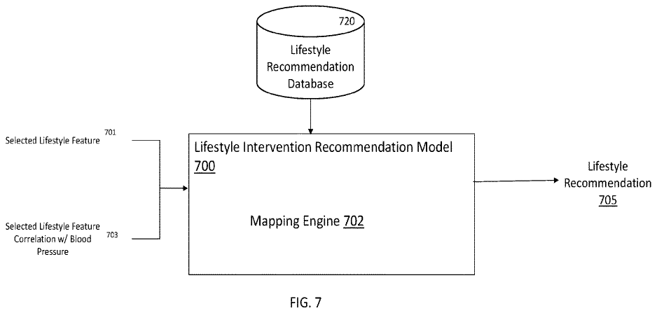

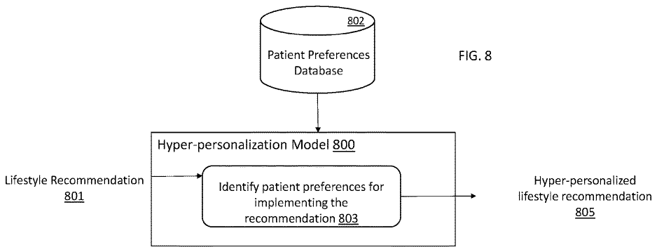

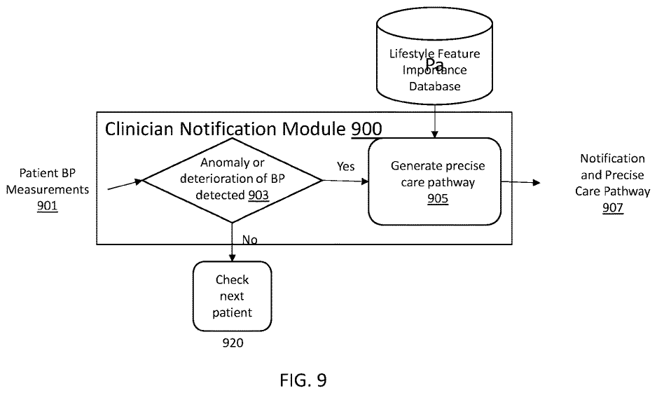

Turning back to FIG. 1, data from user devices 101, 103, electronic health records 107, clinician devices 105, can be transmitted to and received by server 109 via network 110. Server 109 may include one or more components including models and modules configured for a digital lifestyle intervention system for hypertension. For example, server 109 may include a lifestyle feature engineering model 111 (see also FIG. 3), a personal machine learning model 113 (see also FIG. 4), a population model 115 (see also FIG. 5), a lifestyle intervention recommendation model 117 (see also FIGS. 7, 8), and a clinician notification module 119 (see FIG. 9). Interactions between components of the server 109 are illustrated, for example, in FIGS. 2 and FIGS. 6A-6C.

FIG. 2 provides a flow-chart of a method for providing a lifestyle intervention recommendation in accordance with the systems described herein. For example, the process illustrated in FIG. 2 may be implemented by the system 100 illustrated in FIG. 1. As illustrated in FIG. 2, a process 200 may involve the following steps. In a first step 201, a personal machine learning model may be trained using historical user data (see also FIGS. 3 and 4). In a second step 203, the trained personal machine learning model may receive user data. In a third step 205, the trained personal machine learning model may generate an output by applying the trained personal machine learning model to the received user data (see also FIG. 6A-6C). In a fourth step 207, at least one lifestyle recommendation can be generated based on at least one of the generated output from the trained personal machine learning model and output from a population model applied to the received user data (see also FIGS. 5, 6A-6C). In a fifth step 209, the at least one generated lifestyle recommendation can be provided to a user via a user interface. Optionally, in some embodiments, the at least one generated lifestyle recommendation and/or an alert can be provided to a clinician or care team.

FIG. 3 provides a block diagram for a lifestyle feature engineering model 300 analogous to lifestyle feature engineering model 111 of FIG. 1. As illustrated in FIG. 3, a lifestyle feature engineering model 300 may be communicatively coupled to a lifestyle factors database 301. The lifestyle feature engineering model 300 may be configured to generate training data such as historical user data 313 for a personal machine learning model like personal machine learning model 113. The lifestyle feature engineering model 300 may receive user data 311 from one or a plurality of user devices (e.g., user devices 101, 103) and/or databases 301, 303, 305. As discussed above, the user data may include blood pressure data, user lifestyle data, user contextual data, and user compliance data. The received user data may be multi-dimensional and received from a plurality of devices having different configurations. The received user data may have been collected at different frequencies. In some embodiments, the lifestyle feature engineering model 300 may be configured to process the received user data by aligning the received user data based on timestamps and/or other metadata associated with the received user data.

The lifestyle feature engineering model 300 may aggregate, and preprocess the received user data in order to generate historical user data. In some embodiments, the disclosed system may support multiple devices, with each device collecting different lifestyle factors. Accordingly, in some embodiments, the lifestyle feature engineering model 300 may be communicatively coupled to a user device information database 303 that stores user device information. User device information may include a manufacturer, data format, data timing information, data type information, and the like. In some embodiments, the user device information is stored in database 303 along with a user identifier. Using the user device information, the lifestyle feature engineering model 300 may select a subset of the data received from the user devices to generate lifestyle data for a user 307. Blood pressure data received from user devices may be stored separately in a blood pressure database 305 that can be queried for use by the lifestyle feature engineering model 300.

Examples of user data corresponding to user lifestyle data are provided in Table 1. For example, the received user data may include blood pressure data at a frequency of 1-2 readings per day retrieved from a blood pressure cuff. The received user data can also include user lifestyle data such as activity data (e.g., steps, walking/running speed, floors, distance, sedentary time, light activity time, fairly active time, very active time, active energy burned, basal energy burned, standing time, standing frequency, stair ascent speed, stair descent speed, exercise time), sleep data (e.g., sleep duration, bed time, wake up time, light sleep, deep sleep, REM sleep, sleep awareness, respiratory rate), heart rate (HR) data (max active heart rate, mean active heart rate, number of minutes in sedentary HR zone, number of minutes in fatburn HR zone, number of minutes in Cardio HR zone, number of minutes in peak HR zone, sleep HR, sleep HR fluctuations, resting HR, walking HR, HR variation), stress data, mood data, and/or diet data (e.g., alcohol consumption, meat consumption, fruit consumption, vegetable consumption, salt consumption). As shown in Table 1, the user data can also include user contextual data such as medication data including, for example, medication adherence information, and demographics data. The user data can also include user preferences. As shown in Table 1, the user data may be recorded and/or received at varying frequencies. For example, user data can be entered every minute, every hour, daily, 1-2 times daily, weekly, once, and the like. As illustrated, the user data can be obtained from various sources including, for example, a blood pressure cuff, fitness watch and/or mobile application.

TABLE 1
Summary of data collected.

| Category | Signal | Frequency | Source |
|---|---|---|---|
| Blood Pressure | Blood Pressure | 1-2 Readings Daily | Blood Pressure Cuff |
| Activity | Steps | Every Minute | Fitness Watch |
| Activity | Walking/Running Speed | Every Minute | Fitness Watch |
| Activity | Floors | Every Minute | Fitness Watch |
| Activity | Distance | Every minute | Fitness Watch |
| Activity | Sedentary Time | Every Minute | Fitness Watch |
| Activity | Lightly Active Time | Every Minute | Fitness Watch |
| Activity | Fairly Active Time | Every Minute | Fitness Watch |
| Activity | Very Active Time | Every Minute | Fitness Watch |
| Activity | Active Energy Burned | Every Minute | Fitness Watch |
| Activity | Basal Energy Burned | Every Minute | Fitness Watch |
| Activity | Standing Time | Every Minute | Fitness Watch |
| Activity | Standing Frequency | Every Hour | Fitness Watch |
| Activity | Stair Ascent Speed | Every Minute | Fitness Watch |
| Activity | Stair Descent Speed | Every Minute | Fitness Watch |
| Activity | Apple Exercise Time | Every Minute | Fitness Watch |
| Sleep | Sleep Duration | Daily | Fitness Watch |
| Sleep | Bed Time | Daily | Fitness Watch |
| Sleep | Wake Up Time | Daily | Fitness Watch |
| Sleep | Light Sleep | Daily | Fitness Watch |
| Sleep | Deep Sleep | Daily | Fitness Watch |
| Sleep | REM Sleep | Daily | Fitness Watch |
| Sleep | Sleep Awakeness | Daily | Fitness Watch |
| Sleep | Respiratory Rate | Daily | Fitness Watch |
| Heart Rate | Max Active HR | Daily | Fitness Watch |
| Heart Rate | Mean Active HR | Daily | Fitness Watch |
| Heart Rate | Sedentary HR Zone Minutes | Daily | Fitness Watch |
| Heart Rate | Fatburn HR Zone Minutes | Daily | Fitness Watch |
| Heart Rate | Cardio HR Zone Minutes | Daily | Fitness Watch |
| Heart Rate | Peak HR Zone Minutes | Daily | Fitness Watch |
| Heart Rate | Sleep HR | Every Minute | Fitness Watch |
| Heart Rate | Sleep HR Fluctuation | Every Minute | Fitness Watch |
| Heart Rate | Resting HR | Daily | Fitness Watch |
| Heart Rate | Walking HR | Daily | Fitness Watch |
| Heart Rate | Heart Rate Variation | Every Hour | Fitness Watch |
| Stress | Stress | Daily | Mobile App |
| Mood | Mood | Daily | Mobile App |
| Diet | Alcohol Consumption | Daily | Mobile App |
| Diet | Meat Consumption | Daily | Mobile App |
| Diet | Fruit Consumption | Daily | Mobile App |
| Diet | Vegetable Consumption | Daily | Mobile App |
| Diet | Salt Consumption | Daily | Mobile App |
| Medication | Medication Adherence | Daily | Mobile App |
| Demographics | Patient Demographics | Once | Mobile App |
| Preferences | Patient Preferences | Weekly | Mobile App |

The lifestyle feature engineering model 300 may apply feature engineering techniques 309 to the user data received from the user devices and/or stored in the lifestyle factors database 301 and/or blood pressure database 305 to generate training data including historical user blood pressure data and historical user lifestyle data. In some embodiments the historical user blood pressure data and historical user lifestyle data can be time-aligned for use by a machine learning or statistical model (e.g., personal machine learning or statistical model). In some embodiments, the historical user lifestyle data can be aggregated N hours before each timestamped blood pressure, where N can equal 3, 12, 24, 48, 72, etc. In some embodiments, a moving average or exponentially weighted moving average representation of historical user lifestyle data can be time aligned with blood pressure data to generate a feature set designed to have a longer memory or shorter memory depending on the choice of parameters.

FIG. 4 provides a block diagram for a personal machine learning model 400 analogous to personal machine learning model 113 of FIG. 1. The personal machine learning model 400 may be configured to receive training data such as historical user blood pressure data and historical user lifestyle data as generated by a lifestyle feature engineering model 300. The personal machine learning model 400 may be trained to determine a prediction for a user's blood pressure based on their provided lifestyle data.

Historical data can include historical blood pressure data and/or historical user lifestyle data obtained over any suitable period of time (e.g., one month of data, one week of data, one day of data, etc.). Further, the historical data can be from a time period adjacent to the current time, when the personal machine learning model 400 is applied to incoming user data, or from a time period that elapsed some time ago.

In some embodiments, the personal machine learning model 400 may be configured as either a single machine learning model or an ensemble of multiple machine learning models, which may encompass fine-tuned versions of pre-trained models or customized variants of off-the-shelf models. Examples of machine learning models include, but are not limited to, random forest models, gradient boosting models, neural networks, or any combination thereof, potentially adjusted or optimized to enhance predictive performance specific to the individual user's data.

In some embodiments, the personal machine learning model 400 can be trained to output a blood pressure prediction 401 based on an input of blood pressure data and/or user lifestyle data using the random forest model, gradient boosting model, neural network, or the like. For example, the output may be a numeric value, a categorical range, and the like. In some embodiments, the blood pressure prediction may be used in blood pressure trend prediction. For example, blood pressure trend prediction may predict blood pressure over multiple time scales (e.g., one day, one week, etc.) based on an input user lifestyle data.

In some embodiments, the personal machine learning model 400 may be used for lifestyle feature impact analysis 403. For example, using explainable artificial intelligence techniques, the personal machine learning model may be able to identify the subset of user lifestyle data or lifestyle features that are most impactful in reducing a user's blood pressure. Examples of explainable artificial intelligence techniques include Shapley Value Analysis, which calculates the average or weighted average of marginal contributions of each lifestyle feature to the model's predictions, and the like. In some embodiments, the procedure for employing explainable AI with respect to the personal machine learning model 400 (and/or the population model 500 discussed below) involves systematically evaluating the contribution of each feature within the dataset to the prediction outcome, ensuring that the technique is model-agnostic and compatible with various underlying structures of the machine learning model. This process permits a comprehensive understanding of feature influence regardless of the machine learning model employed. In this manner, the disclosed system may be able to determine the impact of lifestyle features on the model's predictions.

In some embodiments, the personal machine learning model 400 can be configured to provide a ranking of lifestyle features based on their impact on the user's blood pressure. The ranking of lifestyle features can be based upon the lifestyle feature impact analysis process which uses explainable AI techniques such as Shapley Analysis. This information can be stored in a lifestyle feature importance database 405. Additionally, in some embodiments, the personal machine learning model 400 may also generate detailed correlation parameters that elucidate the strength and direction of the relationship between each lifestyle feature and blood pressure. For example, the correlation may be generated using one or more statistical correlation analysis techniques including but not limited to Pearson, Spearman or Kendall Analysis techniques. The correlation may be stored in a lifestyle feature correlation database 407, providing a multifaceted view of the interactions between lifestyle and blood pressure.

In some embodiments, the personal machine learning model 400 can include a statistical analysis model. For example, the personal machine learning model 400 may include one or more sub-models configured to apply a deterministic algorithm, regression techniques, heuristic techniques, or other statistical based analysis.

FIG. 5 illustrates a population model 500 analogous to population model 115 of FIG. 1. A population model can be configured to apply one or more statistical techniques to identify lifestyle features and their relative impacts or effect on reducing blood pressure readings. In some embodiments, a population model may be for a corresponding population or group of people having similar age, gender, geographical location, ethnicity, demographics, and the like. A population model 500 may receive lifestyle feature importance data 501a, 501b, 501c, . . . , 501n, etc. generated across one or more people in the population. In some embodiments, the population model 501 can include an aggregator 503 configured to determine the frequency at which a certain lifestyle feature is indicated as an important lifestyle feature across a population. The population model 500 can generate a feature ranking based on the aggregated frequencies 505. A ranked listing of important lifestyle features across the population can be output by the population model 500 and stored in a corresponding population lifestyle features database 507.

In some embodiments, the population model 500 can include a machine learning based population model. For example, in a process analogous to the process illustrated in FIG. 4, a machine learning model can be trained to output indicative of a blood pressure prediction and data characterizing one or more lifestyle features impacting the blood pressure prediction based on a model trained on populations (rather an a specific person). The population based machine learning model can be configured to be trained on data including historical population data including historical user data including historical user blood pressure data and historical user lifestyle data across a plurality of users. In some embodiments, the plurality of users can belong to the same population (e.g., people having similar age, gender, geographical location, ethnicity, demographics, and the like). Further, the population based machine learning model can be configured to be run on data that has been pre-processed in accordance with the feature engineering steps discussed with respect to FIG. 3. For example, historical user device data and historical user lifestyle factors and blood pressure data for a plurality of users belonging to the same population can be used to generate historical population training data.

FIGS. 6A-6C illustrate a process for applying the trained personal machine learning model 400 of FIG. 4 and the population model 500 of FIG. 5 to generate at least one lifestyle recommendation.

As illustrated in FIG. 6A, the system 600 may be used to determine whether a personal machine learning model, a population model, or combination thereof should be used to generate a lifestyle recommendation. For example, the system 600 may receive user data 602 including blood pressure data. The system 600 may first determine whether the received blood pressure data indicates that the user's blood pressure is with a goal or target range, or below a threshold value 601. If the user's blood pressure is within a goal, target range, or below a threshold value, the system 600 may elect to use a personal machine learning model 605 (analogous to personal machine learning model 400) to generate a list of most impactful lifestyle intervention recommendations for a user. If the user's blood pressure is not within a goal, target range, or is consistently above a threshold value, and the user has been enrolled for more than a set enrollment threshold, the system 600 may elect to use a population model. For example, if a user has been enrolled in a program for longer than two months and their blood pressure is not at goal, the population model may be used in generating a list of most impactful lifestyle intervention recommendations for a user. In some embodiments, the blood pressure data can be indicative of a measured blood pressure for the user.

FIG. 6B illustrates a process for generating one or more lifestyle recommendations when a personal machine learning model 605 is elected in the process illustrated in FIG. 6A. As shown in FIG. 6B, the process 600a may access data indicative of which lifestyle interventions may be most impactful in reducing a particular user's blood pressure. For example, the process 600a may utilize data generated during the training of the personal machine learning model 113, 400 and stored as lifestyle feature importance database 405 and the lifestyle feature correlation data 407. In process 600a, the a lifestyle feature may be selected 617 from the lifestyle feature importance database 611 which includes a ranked list of features having the most impact on a user's blood pressure. In a next step, the selected feature can be evaluated to see if it is a clear top feature 619, in that whether the importance of the selected feature in impacting a user's blood pressure is significantly more than the importance of the next ranked feature in impacting a user's blood pressure. In some embodiments, this analysis can use a comparison of Shapley values and/or Pearson coefficients. The impact of a particular lifestyle feature on a user's blood pressure can be determined based at least in part of the correlation data stored in the lifestyle feature correlations database 615 (analogous to lifestyle feature correlation data 407). In some embodiments, the ranked lifestyle features can be iteratively selected until a clear top lifestyle feature is identified 619. In some embodiments, the ranked lifestyle features can be iteratively selected a set number of times (e.g., one time, two times, three times, five times) 621. After a lifestyle feature is selected, at step 623 the process 600a may perform a check to determine whether the selected lifestyle feature is aligned with user indicated preferences and compliance. User preferences and compliance data can be retrieved from database 625. User preferences and compliance data stored in database 625 can be generated from user data including user contextual data and user compliance data received from a user device. In some embodiments, a historical record of past recommendations provided to a user as well as user responses to those recommendations may be used to determine user preferences. For example, a user may indicate using a mobile application whether they were able to follow a recommendation or whether they would prefer to see more or less of a recommendation. The user preference and compliance data may also be indicative of the directionality of lifestyle features, where a user can indicate whether more or less of a particular type of intervention is preferred. The process 600a outputs the selected lifestyle feature 627 and a corresponding correlations with blood pressure 629.

FIG. 6C illustrates a process for generating one or more lifestyle recommendations when a population model 607 is elected in the process illustrated in FIG. 6A. As illustrated in FIG. 6C, a process 600b can be executed by the population model 607. The process 600b illustrates a process for generating lifestyle recommendations based on features determined to be most impactful among a population. For example, the process 600b may utilize feature data 651 stored in the population lifestyle features database 507 which indicates the most impactful lifestyle features for a population. In a first step 653 of the process 600b, the top most ranked lifestyle feature for can be selected. In a second step 655 a check can be performed to determine whether the selected lifestyle feature is aligned with user preferences and user compliance data such as that stored in database 625. A next feature from the population lifestyle feature database 507 can be selected iteratively until a feature aligned with user preferences and compliance is chosen. In a third step 657, a second check as to whether the recommendation has been sent to a user in prior history is performed. If not, that feature is selected. The process 600b outputs the selected lifestyle feature 627 and corresponding correlations with blood pressure 629.

FIG. 7 illustrates a lifestyle intervention recommendation model 700 analogous to lifestyle intervention recommendation model 117 of FIG. 1. The lifestyle intervention recommendation model 700 is configured to generate at least one lifestyle recommendation based on at least one of the generated output from the trained personal machine learning model and output from a population model applied to the received user data. As described above with respect to FIGS. 6A-6C, the trained personal machine learning model and/or the population model can be applied to received user data to generate a selected lifestyle feature (e.g, lifestyle feature 627) and/or a corresponding correlation with blood pressure (e.g., correlation between lifestyle feature and blood pressure 629). The process illustrated in FIG. 7 can be used to generate at least one lifestyle recommendation based on the selected lifestyle feature and/or corresponding correlation with blood pressure. As shown in FIG. 7, the model 700 receives a lifestyle feature 701 and a lifestyle feature correlation with blood pressure 703. The process model 700 includes a mapping engine 702 configured to access a lifestyle recommendation database 720. Using the mapping engine 702 the received lifestyle feature 701 and correlation 703 can be mapped to an actionable lifestyle recommendation 705 which is output by the process. In some embodiments, the mapping engine can include a lookup table. In some embodiments, the actionable lifestyle recommendation 705 can be output to a user interface on a user device.

For example, the correlation between the lifestyle feature and blood pressure indicated by correlation data 703 can indicate the directionality of the relationship. Positive and negative correlations may result in different recommendations. Some lifestyle features are directly actionable and therefore the corresponding lifestyle recommendation is clear. For example, if the selected lifestyle feature is “daily steps”, and the correlation between daily steps and BP is negative, the corresponding lifestyle recommendation will be “increase daily steps”. However, some lifestyle features are not directly actionable and therefore the corresponding recommendation is not clear. For example, the feature “REM sleep” is not directly actionable. The lifestyle feature to recommendation mapping engine is able to map these lifestyle features to clinically meaningful, actionable lifestyle recommendations a user can implement in their daily life.

FIG. 8 provides an illustration of a hyper-personalization process that can be used to further fine-tune lifestyle recommendations generated by the lifestyle intervention recommendation model 700 of FIG. 7. As illustrated in FIG. 8, a hyper-personalization module 800 can receive a lifestyle recommendation 801 as generated by model 700, and retrieve user preferences from a database 802. The hyper-personalization module 800 can be configured to apply user preferences 803 for implementing the recommendation in order to generate hyper-personalized lifestyle recommendations 805 that are individually tailored to the user. In some embodiments, the hyper-personalization process can include a first mode where users are asked how they would prefer to implement a certain lifestyle recommendation. The first mode may encourage the user to focus on their choices. For example, if a given recommendation is to improve sleep hygiene, the hyper-personalization process may ask the user to select their preferred methods to follow the recommendation, including but not limited to: Going to sleep at the same time every night, Avoiding electronic screens 30 minutes before bedtime, Keeping their bedroom pitch black at night, Keeping day time naps below 20 minutes, Avoiding large meals before sleeping, Avoiding caffeine later in the day, Adding some white noise to their bedroom, etc. The user preferences pertaining to a specific recommendation can be stored in a database 802 for future use by the hyper-personalization module 800. During the first mode, the hyper-personalization process can ask the user how they would like to implement a lifestyle recommendation.

After the first mode is completed, after a set number of recommendations have been provided to a user (e.g., 1 recommendation, 5 recommendations, etc.), the hyper-personalization module 800 can operate in a second mode. In the second mode, a previously provided lifestyle recommendation can include hyper-personal recommendations that are based on their previously identified preferences stored in database 802.

One or both of the lifestyle recommendation(s) generated by the lifestyle intervention recommendation model 700 and/or the hyper-personalization model 800 can be provided automatically to a user for a display on a user device. For example, these recommendations can be provided in an automated manner to produce ongoing patient engagement and personalized hypertension management.

User lifestyles are dynamic as people's activities, sleep patterns, dietary habits, etc. are prone to constant change. Due to the dynamic nature of lifestyle, changes in lifestyle patterns can have great impacts on blood pressure, patient context, and preferences. Accordingly, in some embodiments the personal machine learning model 400 and population model 500 can be configured to continuously update and evolve over time in order to provide correct recommendations and ongoing personalized hypertension management. For example, the personal machine learning model 400 can be retrained on a daily, weekly, or monthly basis, etc. to account for shifts in a user's lifestyle. In some embodiments, the described systems and methods can continually query users about their experience with the recommendations provided and their preferences in order to establish a feedback loop between the system and patient resulting in an intelligent machine-human collaboration for managing hypertension. For example, user compliance, user preference data can be updated on a regular basis and the updated data can be used to generate future lifestyle recommendations and/or hyper-personalized recommendations.

As illustrated in FIG. 9, in some embodiments, the disclosed systems can include a clinician notification module 900, analogous to clinician notification module 119 of FIG. 1. As illustrated in FIG. 9, the clinician notification module 900 may receive a patient blood pressure measurement 901. At step 903 the module may determine that the received blood pressure measurement includes an anomaly or is a deterioration of blood pressure management. For example, an anomalous BP reading is defined as lower or higher than certain thresholds such as 90/50 and 180/110 mmHg, respectively. Deterioration of BP can be determined by analyzing the BP trend (e.g., moving average) and triggering a notification if the trend increases by a certain threshold (e.g., 10 mmHg). If there is no anomaly or deterioration detected at step 903, then the system will check the next patient or stop the process. If there is an anomaly or deterioration detected, then the system can notify the clinician and generate a precise care pathway 905 which can be sent to a clinician device, such as clinician device 105 of FIG. 1. A patient care pathway 905 can be configured to address the particular lifestyle factors contributing to the patient's blood pressure anomalies or deterioration. It takes into account the unique lifestyle and health data of the patient, as interpreted by the personal machine learning model. The patient care pathway 905 can provide actionable insights that enable the clinician to intervene effectively and promptly. The determination of a precise care pathway 905 can be generated by identifying the top lifestyle features that can be identified by applying the trained personal machine learning model on user data corresponding to the deterioration period. For example, in some embodiments, the system may identify the top lifestyle features for the deterioration period and weights them based on weekly BP increase, frequency of the feature, and/or proximity to the notification. The top 1-3 weighted lifestyle features can then be used to generate the precise care pathway which informs the clinician which lifestyle factors to investigate for that patient. The clinician notification module 900 can output a notification and/or precise care pathway 907 for use by a clinician device.

The disclosed machine learning based system and remote monitoring devices can be used to help patients more effectively manage their hypertension by interfacing with workers in healthcare systems (including physicians, nurses, clinicians, chronic management care team) by providing a clinician care team with the generated lifestyle recommendations and/or user data recorded and/or received by the system. In some embodiments, the system may include a notification channel where critical and anomaly notifications are sent to the health care workers if a patient's health condition deteriorates.

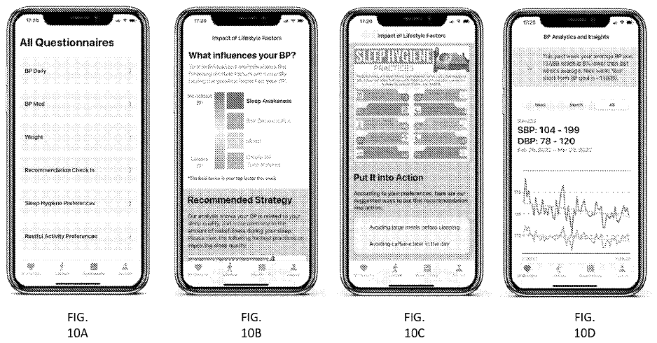

FIGS. 10A-10D provide illustrations of a user interface such as a software application on a mobile device that can be used in accordance with the current subject matter. FIG. 10A provides an illustration of an interactive user interface that is configured to receive information from a user that can input user data regarding their daily activities, medicine, weight recommendation preferences, sleep hygiene preferences, restful activity preferences, and the like.

FIG. 10B provides an illustration of an interactive user interface that provides a user with information as to the lifestyle features most impacting their blood pressure. For example, in some embodiments, the data illustrated in FIG. 10B can be correlated with the data generated by training the personal machine learning model.

FIG. 10C provides an illustration of an interactive user interface that provides a user with actionable lifestyle intervention recommendations that account for their individualized preferences. For example, in some embodiments, the data illustrated in FIG. 10C can be correlated with the data generated by at least one of the lifestyle intervention recommendation model and/or a hyper-personalization model.

FIG. 10D provides an illustration of blood pressure analytics and insights. In some embodiments, the user data and/or a blood pressure prediction generated by the personal machine learning model can be provided to a user using a graphical representation.

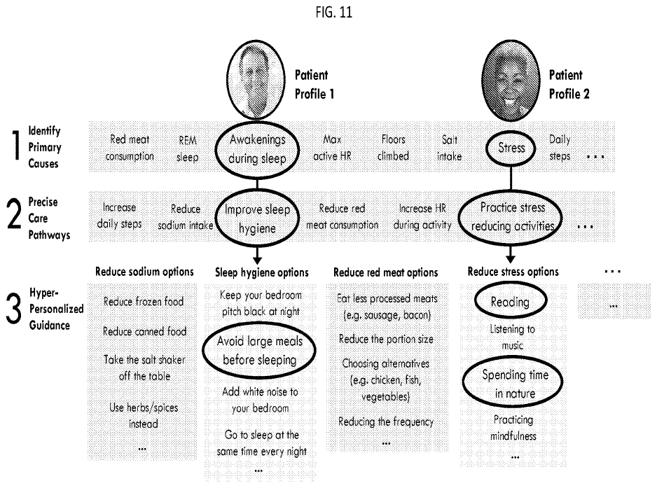

FIG. 11 provides an illustration of lifestyle features, determined precise care pathways and hyper-personalized guidance. For example, when a patient's high blood pressure is attributable to their awakening during sleep, a lifestyle recommendation may be tailored to improve sleep hygiene. A hyper-personalization guidance can provide additional lifestyle intervention recommendations such as avoiding large meals before sleeping. In another example, when a patient's high blood pressure is attributable to their stress, a lifestyle recommendation may be tailored to practice stress reducing activities. A hyper-personalization guidance can provide additional lifestyle intervention recommendations such as reading and spending time in nature.

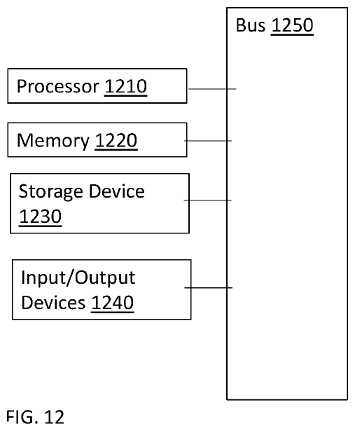

FIG. 12 depicts a block diagram illustrating a computing system 1200 consistent with implementations of the current subject matter. The computing system 1200 may be used to host one or more aspects disclosed herein such as the ML models and processes discloses herein.

As shown in FIG. 12, the computing system 1200 can include a processor 1210, a memory 1220, a storage device 1230, and input/output devices 1240. According to implementations of the current subject matter, a trusted execution environment may be a secure area that may be contained in the processor 1210, or it may be an additional hardware and/or software component. The trusted execution environment may run enclaves to guarantee confidentiality and integrity protection to code and data contained therein, even in an untrusted environment.

The processor 1210, the memory 1220, the storage device 1230, and the input/output devices 1240 can be interconnected via a system bus 1250. The processor 1210 is capable of processing instructions for execution within the computing system 1200. Such executed instructions can implement one or more components of, for example, the trusted server, client devices (parties), and/or the like. In some implementations of the current subject matter, the processor 1210 can be a single-threaded processor. Alternately, the processor 1210 can be a multi-threaded processor. The process may be a multi-core processor have a plurality or processors or a single core processor. The processor 1210 is capable of processing instructions stored in the memory 1220 and/or on the storage device 1230 to display graphical information for a user interface provided via the input/output device 1240.

The memory 1220 is a computer readable medium such as volatile or non-volatile that stores information within the computing system 1200. The memory 1220 can store data structures representing configuration object databases, for example. Although a plurality of databases are discussed herein, it is envisioned that one database or multiple databases storing the data described herein can be used. The storage device 1230 is capable of providing persistent storage for the computing system 1200. The storage device 1230 can be a floppy disk device, a hard disk device, an optical disk device, or a tape device, or other suitable persistent storage means. The input/output device 1240 provides input/output operations for the computing system 1200. In some implementations of the current subject matter, the input/output device 1240 includes a keyboard and/or pointing device. In various implementations, the input/output device 1240 includes a display unit for displaying graphical user interfaces.

According to some implementations of the current subject matter, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A method comprising:
training, based on historical user data, a personal machine learning model to generate an output indicative of a blood pressure prediction and data characterizing one or more lifestyle features impacting the blood pressure prediction, wherein the historical user data comprises historical user blood pressure data and historical user lifestyle data for a user;
receiving, by the trained personal machine learning model, user data, wherein the user data further comprises blood pressure data for the user;
generating, by the trained personal machine learning model, an output by applying the trained personal machine learning model to the received user data;
generating at least one lifestyle recommendation based on at least one of the generated output from the trained personal machine learning model and output from a population model applied to the received user data; and
providing at least one of the at least one generated lifestyle recommendation, and the output indicative of the blood pressure prediction, and the data characterizing one or more lifestyle features impacting the blood pressure prediction to the user via a user interface.

2. The method of claim 1, wherein the user data further comprises at least one of user lifestyle data, user contextual data, and user compliance data.

3. The method of claim 1, wherein the trained machine learning model further comprises at least one of a random forest model, a gradient boosting model, and a neural network.

4. The method of claim 1, wherein generating the at least one recommendation for a lifestyle modification further comprises:
identifying a lifestyle recommendation from a lifestyle recommendation database communicatively coupled to the trained personal machine learning model, based on the data characterizing one or more lifestyle features impacting the blood pressure prediction.

5. The method of claim 1, wherein the data characterizing one or more lifestyle features impacting the blood pressure prediction further comprises at least one of ranking data for the one or more lifestyle features impacting the blood pressure prediction, and correlation data for the one or more lifestyle features impacting the blood pressure prediction.

6. The method of claim 1, wherein the population model is configured to generate the at least one recommendation based on an aggregation of feature importance data based on a historical population.

7. The method of claim 1, wherein the population model comprises a machine learning model trained on historical population data comprising historical user data comprising historical user blood pressure data and historical user lifestyle data for a plurality of users.

8. The method of claim 1, further comprising:
receiving at least one of user preference data and user compliance data; and
modifying the generated at least one recommendation based on at least one of user preference data and user compliance data.

9. The method of claim 1, further comprising:
providing at least one of the at least one recommendation or sensor data to a clinician via a clinician user interface.

10. The method of claim 1, wherein the user data further comprises lifestyle data.

11. The method of claim 1, receiving at least one of blood pressure data, compliance data, preference data, or an updated user lifestyle data; and
updating the trained machine learning model.

12. (canceled)

13. (canceled)

14. (canceled)

15. (canceled)

16. A system, comprising:
at least one data processor; and
at least one memory storing instructions which, when executed by the at least one data processor, result in operations comprising:
training, based on historical user data, a personal machine learning model to generate an output indicative of a blood pressure prediction and data characterizing one or more lifestyle features impacting the blood pressure prediction, wherein the historical user data comprises historical user blood pressure data and historical user lifestyle data for a user;
receiving, by the trained personal machine learning model, user data, wherein the user data further comprises blood pressure data for the user;
generating, by the trained personal machine learning model, an output by applying the trained personal machine learning model to the received user data;
generating at least one lifestyle recommendation based on at least one of the generated output from the trained personal machine learning model and output from a population model applied to the received user data; and
providing at least one of the at least one generated lifestyle recommendation, and the output indicative of the blood pressure prediction, and the data characterizing one or more lifestyle features impacting the blood pressure prediction, to the user via a user interface.

17. The system of claim 16, wherein the user data further comprises at least one of user lifestyle data, user contextual data, and user compliance data.

18. The system of claim 16, wherein the trained machine learning model further comprises at least one of a random forest model and a gradient boosting model.

19. The system of claim 16, wherein generating the at least one recommendation for a lifestyle modification further comprises:
identifying a lifestyle recommendation from a lifestyle recommendation database communicatively coupled to the at least one processor, based on the data characterizing one or more lifestyle features impacting the blood pressure prediction.

20. The system of claim 16, wherein the data characterizing one or more lifestyle features impacting the blood pressure prediction further comprises at least one of ranking data for the one or more lifestyle features impacting the blood pressure prediction, and correlation data for the one or more lifestyle features impacting the blood pressure prediction.

21. The system of claim 16, wherein the population model further comprises at least one of: an aggregator configured to generate the at least one recommendation based on an aggregation of feature importance data based on a historical population; or
a machine learning model trained on historical population data comprising historical user data comprising historical user blood pressure data and historical user lifestyle data for a plurality of users.

22. The system of claim 16, wherein the operations further comprises:
receiving, by the at least one processor, at least one of user preference data and user compliance data; and
modifying, by the at least one processor, the generated at least one recommendation based on at least one of user preference data and user compliance data.

23. The system of claim 16, wherein the operations further comprises:
receiving at least one of blood pressure data, compliance data, preference data, or an updated user lifestyle data; and
updating the trained machine learning model.
